(19) United States
(12) Patent Application Publication
(10) Pub. No.: US 2026/0055080 A1
(43) Pub. Date: Feb. 26, 2026

(54) NOVEL SUBSTITUTED QUINOLINE AND TETRAHYDRONAPHTHALENE CARBOXYLIC ACID DERIVATIVES AND THERAPEUTIC USES THEREOF

(71) Applicant: SANOFI, Paris (FR)

(72) Inventor: Youssef El-Ahmad, Paris (FR)

(21) Appl. No.: 19/105,432

(22) PCT Filed: Aug. 24, 2023
(86) PCT No.: PCT/EP2023/073221

§ 371 Date: Feb. 21, 2025

(30) Foreign Application Priority Data
Aug. 25, 2022 (EP) 22306254.8

(51) Int. Cl. A61K 31/4709; A61K 31/24; A61K 31/255; A61P 5/32; C07C 229/52; C07C 309/65; C07D 401/12

(52) U.S. Cl. CPC A61K 31/4709; A61K 31/24; A61K 31/255; A61P 5/32; C07C 229/52; C07C 309/65; C07D 401/12

(57) ABSTRACT

Disclosed herein is a compound, or a pharmaceutically acceptable salt thereof, in particular hydrochloride salt thereof, characterized in that said compound is selected from the following compounds: -4-(4-((1-(3-fluoropropyl)azetidin-3-yl)oxy)benzoyl)-3-(4-(trifluoromethyl)phenyl)quinoline-7-carboxylic acid (1), -3-(2-fluoro-4-(trifluoromethyl)phenyl)-4-(4-(2-(3-(fluoromethyl)azetidin-1-yl)ethoxy)benzoyl)quinoline-7-carboxylic acid (2), and -(R)-6-(2-(ethyl(4-(2-(ethylamino)ethyl)benzyl)amino)-4-methoxyphenyl)-5,6,7,8-tetrahydronaphthalene-2-carboxylic acid (3). Further disclosed are process for preparing the same, pharmaceutical compositions comprising them as well as said compounds of formula (I) for use as an inhibitor and degrader of estrogen receptors, in particular in the treatment of ovulatory dysfunction, cancer, endometriosis, osteoporosis, benign prostatic hypertrophy or inflammation.

Disclosed herein are novel substituted quinoline and tetrahydronaphthalene carboxylic acid derivatives, the processes for their preparation, as well as the therapeutic uses thereof, in particular as anticancer agents via selective antagonism and degradation of estrogen receptors.

The Estrogen Receptors (ER) belong to the steroid/nuclear receptor superfamily involved in the regulation of eukaryotic gene expression, cellular proliferation and in target tissues. ERs are in two forms: the estrogen receptor alpha (ERα) and the estrogen receptor beta (ERβ) respectively encoded by the ESR1 and the ESR2 genes. ERα and ERβ are ligand-activated transcription factors which are activated by the hormone estrogen (the most potent estrogen produced in the body is 17β-estradiol). In the absence of hormone, ERs are largely located in the cytosol of the cell. When the hormone estrogen binds to ERs, ERs migrate from the cytosol to the nucleus of the cell, form dimers and then bind to specific genomic sequences called Estrogen Response Elements (ERE). The DNA/ER complex interacts with co-regulators to modulate the transcription of target genes.

ERα is mainly expressed in reproductive tissues such as uterus, ovary, breast, bone and white adipose tissue. Abnormal ERα signaling leads to development of a variety of diseases, such as cancers, metabolic and cardiovascular diseases, neurodegenerative diseases, inflammation diseases and osteoporosis.

ERα is expressed in not more than 10% of normal breast epithelium but approximately 50-80% of breast tumors. Such breast tumors with high level of ERα are classified as ERα-positive breast tumors. The etiological role of estrogen in breast cancer is well established and modulation of ERα signaling remains the mainstay of breast cancer treatment for the majority ERα-positive breast tumors. Currently, several strategies for inhibiting the estrogen axis in breast cancer exist, including: 1-blocking estrogen synthesis by aromatase inhibitors that are used to treat early and advanced ERα-positive breast cancer patients; 2-antagonizing estrogen ligand binding to ERα by tamoxifen which is used to treat ERα-positive breast cancer patients in both pre- and post-menopausal setting; 3-antagonizing and downregulating ERα levels by fulvestrant, which is used to treat breast cancer in patients that have progressed despite endocrine therapies such as tamoxifen or aromatase inhibitors.

Although these endocrine therapies have contributed enormously to reduction in breast cancer development, about more than one-third of ERα-positive patients display de novo resistance or develop resistance over time to such existing therapies. Several mechanisms have been described to explain resistance to such hormone therapies. For example, hypersensitivity of ERα to low estrogen level in treatment with aromatase inhibitors, the switch of tamoxifen effects from antagonist to agonist effects in tamoxifen treatments or multiple growth factor receptor signaling pathways. Acquired mutations in ERα occurring after initiation of hormone therapies may also play a role in treatment failure and cancer progression. Certain mutations in ERα, particularly those identified in the Ligand Binding Domain (LBD), result in the ability to bind to DNA in the absence of ligand and confer hormone independence in cells harboring such mutant receptors.

Most of the endocrine therapy resistance mechanisms identified rely on ERα-dependent activity. One of the new strategies to counterforce such resistance is to shut down the ERα signaling by removing ERα from the tumor cells using Selective Estrogen Receptors Degraders (SERDs). Clinical and preclinical data showed that a significant number of the resistance pathways can be circumvented by the use of SERDs.

There is still a need to provide SERDs with good degradation efficacy.

Documents WO2017/140669 and WO2018/091153 disclose some substituted 6,7-dihydro-5H-benzo[7]annulene compounds and substituted N-(3-fluoropropyl)-pyrrolidine derivatives useful as SERDs.

The inventors have now found novel compounds able to selectively antagonize and degrade the estrogen receptors (SERDs compounds), for use in cancer treatment.

Disclosed herein are compounds, or pharmaceutically acceptable salts thereof, in particular hydrochloride salt thereof, selected from:
4-(4-((1-(3-fluoropropyl)azetidin-3-yl)oxy)benzoyl)-3-(4-(trifluoromethyl)phenyl)quinoline-7-carboxylic acid (1),
3-(2-fluoro-4-(trifluoromethyl)phenyl)-4-(4-(2-(3-(fluoromethyl) azetidin-1-yl)ethoxy)benzoyl)quinoline-7-carboxylic acid (2), and
(R)-6-(2-(ethyl(4-(2-(ethylamino)ethyl)benzyl)amino)-4-methoxyphenyl)-5,6,7,8-tetrahydronaphthalene-2-carboxylic acid (3).

Another embodiment is a compound, or pharmaceutically acceptable salt thereof, in particular hydrochloride salt thereof, selected from:
4-(4-((1-(3-fluoropropyl)azetidin-3-yl)oxy)benzoyl)-3-(4-(trifluoromethyl)phenyl)quinoline-7-carboxylic acid (1), and
(R)-6-(2-(ethyl(4-(2-(ethylamino)ethyl)benzyl)amino)-4-methoxyphenyl)-5,6,7,8-tetrahydronaphthalene-2-carboxylic acid (3).

Said compounds (1), (2) or (3), in particular compounds (1) and (3), can contain one or more asymmetric carbon atoms. They may therefore exist in the form of enantiomers.

Said compounds (1), (2) or (3), in particular compounds (1) and (3), may be present as well under tautomer forms.

Said compounds (1), (2) or (3), in particular compounds (1) and (3), may exist in the form of bases, acids, zwitterion or of addition salts with acids or bases. Hence, herein are provided said compounds or pharmaceutically acceptable salts thereof.

These salts may be prepared with pharmaceutically acceptable acids or bases, although the salts of other acids or bases useful, for example, for purifying or isolating said compounds are also provided.

Among suitable salts of said compounds, hydrochloride may be cited.

Another embodiment is a compound selected from the above lists, or a pharmaceutically acceptable salt thereof, for use in therapy, especially as an inhibitor and degrader of estrogen receptors.

Another embodiment is a compound selected from the above lists, or a pharmaceutically acceptable salt thereof, for use in the treatment of cancer, especially breast cancer.

Another embodiment is a method of inhibiting and degrading estrogen receptors, comprising administering to a subject in need thereof, in particular a human, a therapeutically effective amount of a compound selected from the above lists, or a pharmaceutically acceptable salt thereof.

Another embodiment is a method of treating ovulatory dysfunction, cancer, endometriosis, osteoporosis, benign prostatic hypertrophy or inflammation, comprising administering to a subject in need thereof, in particular a human, a therapeutically effective amount of a compound selected from the above lists, or a pharmaceutically acceptable salt thereof.

Another embodiment is a method of treating cancer, comprising administering to a subject in need thereof, in particular a human, a therapeutically effective amount of a compound selected from the above lists, or a pharmaceutically acceptable salt thereof.

Another embodiment is a pharmaceutical composition comprising as active principle an effective dose of a compound selected from the above lists, or a pharmaceutically acceptable salt thereof, and also at least one pharmaceutically acceptable excipient.

The compounds as provided herein can be prepared by the following processes.

The compounds as provided herein are synthesized using techniques and materials described below or otherwise known by the skilled person in the art. In addition, solvents, temperatures and other reaction conditions presented below may vary as deemed appropriate to the skilled person in the art.

The following abbreviations and empirical formulae are used:

| | |
|---|---|
| NH₄Cl | Ammonium chloride |
| CO | Carbon monoxide |
| DCM | Dichloromethane |
| DIEA | N,N-Diisopropylethylamine |
| DMF | N,N-dimethylformamide |
| DMAP | Dimethylaminopyridine |
| DMSO | Dimethyl sulfoxide |
| LiOH | Lithium hydroxide |
| MeOH | Methanol |
| MgSO₄ | Magnesium sulfate |
| NaH | Sodium hydride |
| THF | Tetrahydrofuran |
| RT | Room temperature |

The ¹H NMR Spectra at 400 and 500 MHz were performed on a Bruker Avance DRX-400 and Bruker Avance DPX-500 spectrometer, respectively, with the chemical shifts (8 in ppm) in the solvent dimethyl sulfoxide-d6 (d6-DMSO) referenced at 2.5 ppm at a temperature of 303 K. Coupling constants (J) are given in Hertz.

The liquid chromatography/mass spectra (LC/MS) were obtained on a UPLC Acquity Waters instrument, light scattering detector Sedere and SQD Waters mass spectrometer using UV detection DAD 210-400 nm and flash Acquity UPLC CSH C18 1.7 μm, dimension 2.1×30 mm, mobile phase H₂O+0.1% HCO₂H/CH₃CN+0.1% HCO₂H.

The following tables 1a and 1b comprises respectively specific compounds of as provided herein (name and structure) in accordance with the present disclosure as well their characterization (¹H NMR and liquid chromatography/mass).

TABLE 1a
(the first column “Ex” corresponds to the compound and example number)

| Ex | Structure | Name |
|---|---|---|
| 1 | 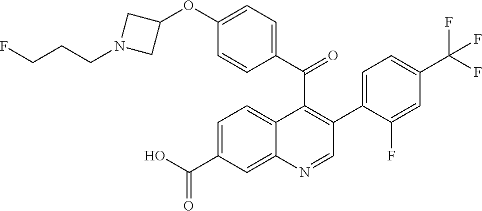 [SMILES: O=C(O)c1ccc2c(C(=O)c3ccc(OC4CN(CCCF)C4)cc3)c(-c3ccc(C(F)(F)F)cc3F)cnc2c1] | 4-(4-((1-(3- fluoropropyl)azetidin-3- yl)oxy)benzoyl)-3-(4- (trifluoromethyl)phenyl) quinoline-7-carboxylic acid |
| 2 | 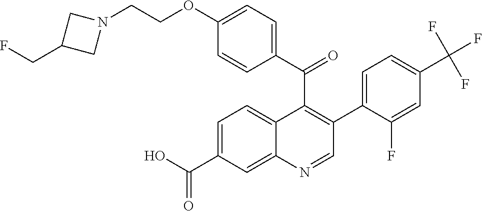 [SMILES: O=C(O)c1ccc2c(C(=O)c3ccc(OCCN4CC(CF)C4)cc3)c(-c3ccc(C(F)(F)F)cc3F)cnc2c1] | 3-(2-fluoro-4- (trifluoromethyl)phenyl)-4- (4-(2-(3- (fluoromethyl)azetidin-1- yl)ethoxy)benzoyl)quinoline- 7-carboxylic acid |
| 3 | 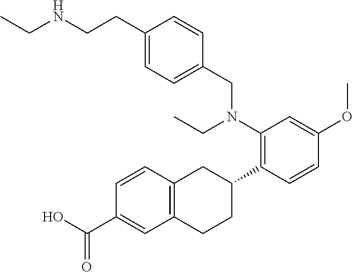 [SMILES: CCNCCc1ccc(CN(CC)c2cc(OC)ccc2[C@@H]2CCc3cc(C(=O)O)ccc3C2)cc1] | (R)-6-(2-(ethyl(4-(2- (ethylamino)ethyl)benzyl)amino)- 4-methoxyphenyl)- 5,6,7,8- tetrahydronaphthalene-2- carboxylic acid |

TABLE 1b

| | | MASS: |
|---|---|---|
| | | LC/MS (m/z, |
| Ex | NMR | MH+): |
| 1 | ¹H NMR (500 MHz, DMSO) δ 12.11 (br, 1H) 9.08 (d, J = | 571 |
| | 2 Hz, 1H), 8.71 (s, 1H), 8.14 (dd, J = 9, 2 Hz, 1H), | |
| | 7.77 (dd, J = 10, 2 Hz, 1H), 7.64-7.51 (m, 5H), 6.92- | |
| | 6.80 (m, 2H), 4.85 (t, J = 6 Hz, 1H), 4.43 (dt, J = 47, 6 | |
| | Hz, 2H), 3.69 (m, 2H), 2.96-2.88 (m, 2H), 1.64 (dt, J = | |
| | 25, 7 Hz, 2H) (2 H under DMSO) | |
| 2 | 1H NMR (400 MHz, DMSO-d6, 30° C.) δ ppm 2.69- | 571 |
| | 2.78 (m, 1 H), 2.78 (br t, J = 5 Hz, 2 H), 3.06 (br t, J = 7 | |
| | Hz, 2 H), 3.37 (br t, J = 7 Hz, 2 H), 4.00 (t, J = 5 Hz, 2 H), | |
| | 4.49 (dd, J = 48, 6 Hz, 2 H), 6.94 (d, J = 9 Hz, 2 H), 7.50- | |
| | 7.64 (m, 4 H), 7.68 (d, J = 9 Hz, 1 H), 7.78 (d, J = 9 Hz, 1 | |
| | H), 8.11 (dd, J = 9, 2 Hz, 1 H), 8.72 (d, J = 1 Hz, 1 H), 9.15 | |
| | (d, J = 1 Hz, 1 H) | |
| 3 | 1H NMR (400 MHz, DMSO-d6, 30° C.) δ ppm 0.82 (t, J = 7 Hz, | 487 |
| | 3 H), 1.01 (t, J = 7 Hz, 3 H), 1.45-1.56 (m, 1 H), 1.59-1.78 | |
| | (m, 1 H), 2.52-2.95 (m, 12 H), 3.44-3.59 (m partilally | |
| | hidden, 1 H), 3.66 (s, 3 H), 3.86-3.97 (m, 2 H), 6.63 (dd, J = 9, | |
| | 3 Hz, 1 H), 6.79 (d, J = 3 Hz, 1 H), 6.98 (d, J = 8 Hz, 1 H), 7.00- | |
| | 7.04 (m, 2 H), 7.04-7.08 (m, 2 H), 7.10 (d, J = 9 Hz, 1 H), | |
| | 7.58 (dd, J = 8, 1 Hz, 1 H), 7.63 (br s, 1 H) | |

Herein is furthermore provided a compound, or any of its pharmaceutically acceptable salt, selected from

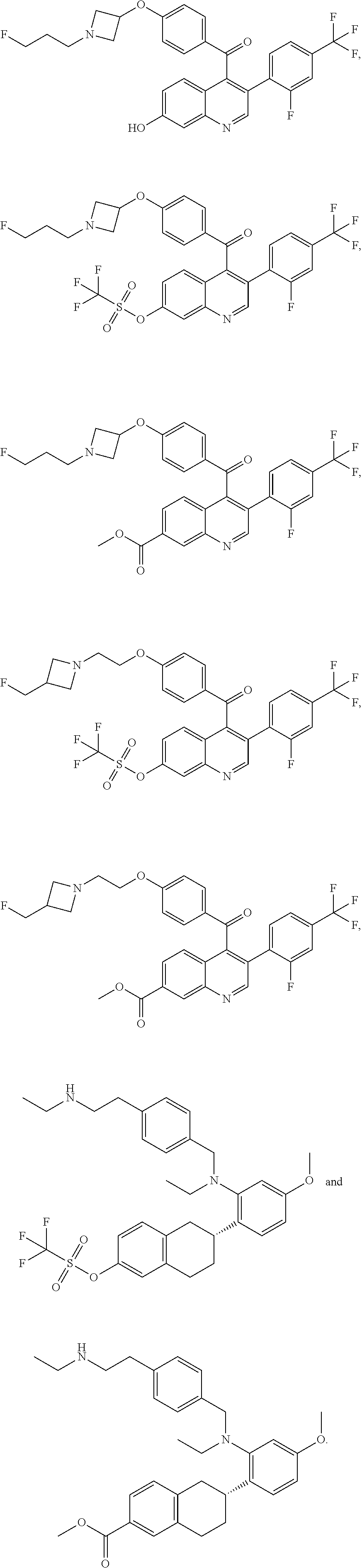

The examples which follow describe the preparation of the compounds as provided herein. The numbers of the compounds exemplified below match those given in the Table 1 above. All reactions are performed under inert atmosphere, unless otherwise stated.

In the following examples, when the source of the starting products is not specified, it should be understood that said products are known compounds.

### EXAMPLES

### Example 1:4-(4-((1-(3-Fluoropropyl)azetidin-3-yl)oxy)benzoyl)-3-(4-(trifluoromethyl)phenyl)quinoline-7-carboxylic Acid

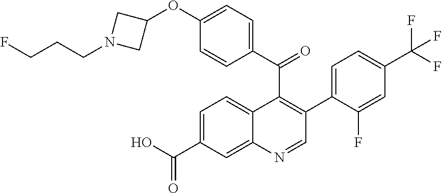 [SMILES: O=C(O)c1ccc2c(C(=O)c3ccc(OC4CN(CCCF)C4)cc3)c(-c3ccc(C(F)(F)F)cc3F)cnc2c1]

### Step 1: (4-((1-(3-Fluoropropyl)azetidin-3-yl)oxy)phenyl)(7-hydroxy-3-(4-(trifluoromethyl)phenyl)quinolin-4-yl)methanone

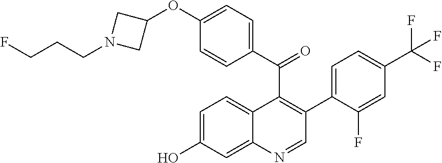 [SMILES: O=C(c1ccc(OC2CN(CCCF)C2)cc1)c1c(-c2ccc(C(F)(F)F)cc2F)cnc2cc(O)ccc12]

NaH 60% (0.23 g, 5.8 mmol) was added to a mixture of 3-[2-fluoro-4-(trifluoromethyl)phenyl]-4-(4-fluorobenzoyl) quinolin-7-ol (prepared according to WO 2020/014440) (0.5 g, 1.2 mmol) and 1-(3-fluoropropyl)azetidin-3-ol (commercially available) (0.33 g, 2.5 mmol) in DMF (6 ml). The mixture was heated at 40° C. for 2 h. After cooling to RT, the crude mixture was concentrated under reduced pressure. To the residue obtained, DCM (20 ml) and water (10 ml) were added. After decantation, the organic phase was dried over MgSO₄, filtered and concentrated under reduced pressure and the residue obtained was purified by flash chromatography eluting with a gradient of MeOH in DCM from 100/00 to 98/02 to give 231 mg (37%) of (4-((1-(3-fluoropropyl)azetidin-3-yl)oxy)phenyl)(7-hydroxy-3-(4-(trifluoromethyl)phenyl)quinolin-4-yl)methanone. LC/MS (m/z, MH+): 543

### Step 2:4-(4-((1-(3-Fluoropropyl)azetidin-3-yl)oxy)benzoyl)-3-(4-(trifluoromethyl)phenyl)quinolin-7-yl trifluoromethanesulfonate

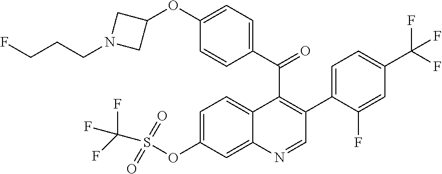 [SMILES: O=C(c1ccc(OC2CN(CCCF)C2)cc1)c1c(-c2ccc(C(F)(F)F)cc2F)cnc2cc(OS(=O)(=O)C(F)(F)F)ccc12]

Triflic anhydride (144 mg, 86 μL, 0.51 mmol) was added to a mixture of (4-((1-(3-fluoropropyl)azetidin-3-yl)oxy)phenyl) (7-hydroxy-3-(4-(trifluoromethyl)phenyl)quinolin-4-yl)methanone (231 mg, 0.43 mmol) and DMAP (104 mg, 0.85 mmol) in DCM (15 ml) cooled down to −10° C. The reaction mixture was stirred at −10° C. for 1 h. The crude mixture was quenched with an aqueous solution of NH₄Cl (5 ml). After decantation, the organic phase was washed with water (5 ml), dried over MgSO₄, filtered and concentrated under reduced pressure to give 171 mg (crude) of 4-(4-((1-(3-fluoropropyl)azetidin-3-yl)oxy)benzoyl)-3-(4-(trifluoromethyl)phenyl)quinolin-7-yl trifluoromethanesulfonate used as such in the next step.

LC/MS (m/z, MH+): 675

### Step 3: Methyl 4-(4-((1-(3-fluoropropyl)azetidin-3-yl)oxy)benzoyl)-3-(4-(trifluoromethyl)phenyl)quinoline-7-carboxylate

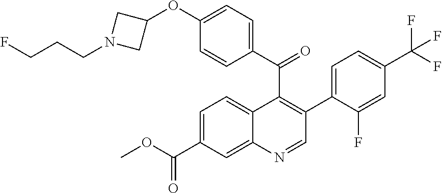 [SMILES: COC(=O)c1ccc2c(C(=O)c3ccc(OC4CN(CCCF)C4)cc3)c(-c3ccc(C(F)(F)F)cc3F)cnc2c1]

In a stainless steel bomb, a mixture of 4-(4-((1-(3-fluoropropyl)azetidin-3-yl)oxy)benzoyl)-3-(4-(trifluoromethyl)phenyl)quinolin-7-yl trifluoromethanesulfonate (171 mg, 0.25 mmol), DMAP (93 mg, 0.76 mmol), palladium diacetate (29 mg, 0.13 mmol) and 1,3-bis(diphenylphosphino)propane (63 mg, 0.15 mmol) in MeOH (12 ml) was submitted to 20 bar pressure of CO and heated at 100° C. for 23 h. The crude solution was concentrated under reduced pressure and diluted in DCM (20 ml) and water (10 ml). After decantation, the organic phase was dried over MgSO₄, filtered and concentrated under reduced pressure. The residue obtained was purified by flash chromatography eluting with a gradient of MeOH in DCM from 100/00 to 98/02 to give 86 mg (58%) of methyl 4-(4-((1-(3-fluoropropyl)azetidin-3-yl)oxy)benzoyl)-3-(4-(trifluoromethyl)phenyl)quinoline-7-carboxylate.

LC/MS (m/z, MH+): 585

### Step 4:4-(4-((1-(3-Fluoropropyl)azetidin-3-yl)oxy)benzoyl)-3-(4-(trifluoromethyl)phenyl)quinoline-7-carboxylic acid

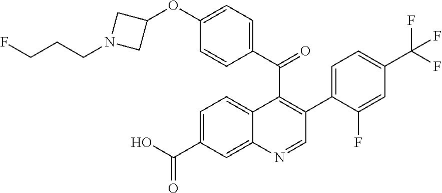 [SMILES: O=C(O)c1ccc2c(C(=O)c3ccc(OC4CN(CCCF)C4)cc3)c(-c3ccc(C(F)(F)F)cc3F)cnc2c1]

A mixture of methyl 3-[2-fluoro-4-(trifluoromethyl)phenyl]-4-(4-{[1-(3-fluoropropyl)azetidin-3-yl]oxy}benzoyl)quinoline-7-carboxylate (86 mg, 0.15 mmol) and LiOH (78 mg, 3.2 mmol) in a mixture of THF (4 ml) and water (4 ml) was stirred at 60° C. for 30 minutes. After cooling to RT, acetic acid (0.18 mL, 3.18 mmol) was added and the mixture was concentrated under reduced pressure. The residue obtained was purified by flash chromatography eluting with a gradient of MeOH in DCM from 100/00 to 95/05 to give 45 mg (54%) of 4-(4-((1-(3-fluoropropyl)azetidin-3-yl)oxy)benzoyl)-3-(4-(trifluoromethyl)phenyl)quinoline-7-carboxylic acid.

### Example 2:3-(2-Fluoro-4-(trifluoromethyl)phenyl)-4-(4-(2-(3-(fluoromethyl) azetidin-1-yl)ethoxy)benzoyl)quinoline-7-carboxylic acid

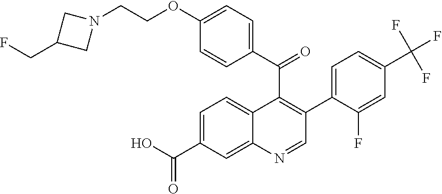 [SMILES: O=C(O)c1ccc2c(C(=O)c3ccc(OCCN4CC(CF)C4)cc3)c(-c3ccc(C(F)(F)F)cc3F)cnc2c1]

### Step 1:4-(4-(2-(3-(Fluoromethyl) azetidin-1-yl)ethoxy)benzoyl)-3-(4-(trifluoromethyl)phenyl)quinolin-7-yl trifluoromethanesulfonate

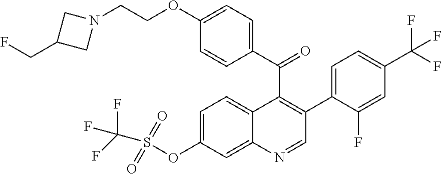 [SMILES: O=C(c1ccc(OCCN2CC(CF)C2)cc1)c1c(-c2ccc(C(F)(F)F)cc2F)cnc2cc(OS(=O)(=O)C(F)(F)F)ccc12]

Step 1 of Example 2 was prepared following a similar procedure to that of step 2 of Example 1 from (4-(2-(3-(fluoromethyl) azetidin-1-yl) ethoxy)phenyl) (7-hydroxy-3-(4-(trifluoromethyl)phenyl)quinolin-4-yl) methanone (prepared according to WO 2020/014440) and triflic anhydride to give 200 mg (crude) of 4-(4-(2-(3-(fluoromethyl) azetidin-1-yl)ethoxy)benzoyl)-3-(4-(trifluoromethyl)phenyl)quinolin-7-yl trifluoromethanesulfonate used as such in the next step.

LC/MS (m/z, MH+): 675

### Step 2: Methyl 4-(4-(2-(3-(fluoromethyl) azetidin-1-yl)ethoxy)benzoyl)-3-(4-(trifluoromethyl)phenyl)quinoline-7-carboxylate

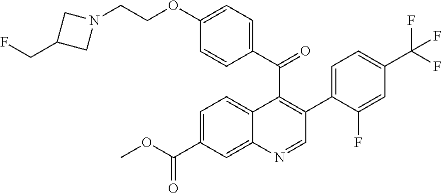 [SMILES: COC(=O)c1ccc2c(C(=O)c3ccc(OCCN4CC(CF)C4)cc3)c(-c3ccc(C(F)(F)F)cc3F)cnc2c1]

In a stainless steel bomb, a mixture of 4-(4-(2-(3-(fluoromethyl) azetidin-1-yl)ethoxy)benzoyl)-3-(4-(trifluoromethyl)phenyl)quinolin-7-yl trifluoromethanesulfonate (200 mg, 0.30 mmol), DIEA (79 mg, 0.61 mmol), [1,1′-bis(diphenylphosphino)ferrocene]dichloropalladium(II) (15 mg, 0.02 mmol) and 1,3-bis(diphenylphosphino)propane (63 mg, 0.15 mmol) in MeOH (2 ml) and DMF (4 ml) was submitted to 5 bar pressure of CO and heated at 70° C. for 5 h. The crude solution filtered over celite and the filtrate was concentrated under reduced pressure. To the residue obtained, DCM (10 ml) and water (10 ml) were added. After decantation, the organic phase was dried over MgSO₄, filtered and concentrated under reduced pressure. The residue obtained was purified by flash chromatography eluting with a gradient of MeOH in DCM from 100/00 to 95/05 to give 95 mg (55%) of methyl 4-(4-(2-(3-(fluoromethyl) azetidin-1-yl)ethoxy)benzoyl)-3-(4-(trifluoromethyl)phenyl)quinoline-7-carboxylate.

LC/MS (m/z, MH+): 585

### Step 3:3-(2-Fluoro-4-(trifluoromethyl)phenyl)-4-(4-(2-(3-(fluoromethyl) azetidin-1-yl)ethoxy)benzoyl)quinoline-7-carboxylic acid

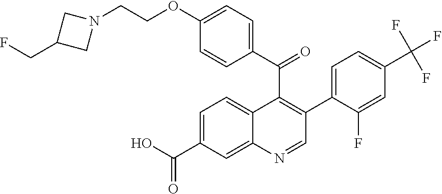 [SMILES: O=C(O)c1ccc2c(C(=O)c3ccc(OCCN4CC(CF)C4)cc3)c(-c3ccc(C(F)(F)F)cc3F)cnc2c1]

Step 3 of Example 2 was prepared following a similar procedure to that of step 4 of Example 1 from methyl 4-(4-(2-(3-(fluoromethyl) azetidin-1-yl)ethoxy)benzoyl)-3-(4-(trifluoromethyl)phenyl)quinoline-7-carboxylate to give 71 mg (77%) of 3-(2-fluoro-4-(trifluoromethyl)phenyl)-4-(4-(2-(3-(fluoromethyl) azetidin-1-yl)ethoxy)benzoyl)quinoline-7-carboxylic acid.

### Example 3: (R)-6-(2-(Ethyl(4-(2-(ethylamino)ethyl)benzyl)amino)-4-methoxyphenyl)-5,6,7,8-tetrahydronaphthalene-2-carboxylic acid

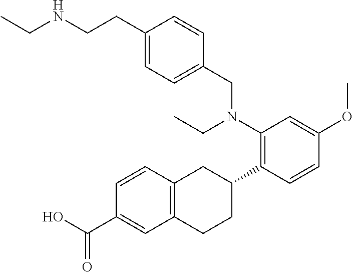 [SMILES: CCNCCc1ccc(CN(CC)c2cc(OC)ccc2[C@@H]2CCc3cc(C(=O)O)ccc3C2)cc1]

### Step 1: (R)-6-(2-(Ethyl(4-(2-(ethylamino)ethyl)benzyl)amino)-4-methoxyphenyl)-5,6,7,8-tetrahydronaphthalen-2-yl trifluoromethanesulfonate

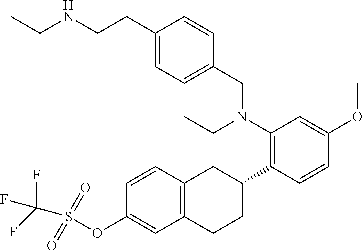 [SMILES: CCNCCc1ccc(CN(CC)c2cc(OC)ccc2[C@@H]2CCc3cc(OS(=O)(=O)C(F)(F)F)ccc3C2)cc1]

Step 1 of Example 3 was prepared following a similar procedure to that of step 2 of Example 1 from (R)-6-(2-(ethyl(4-(2-(ethylamino)ethyl)benzyl)amino)-4-methoxyphenyl)-5,6,7,8-tetrahydronaphthalen-2-ol (prepared according to EP 1557288) and triflic anhydride to give 330 mg (crude) of (R)-6-(2-(ethyl(4-(2-(ethylamino)ethyl)benzyl)amino)-4-methoxyphenyl)-5,6,7,8-tetrahydronaphthalen-2-yl trifluoromethanesulfonate used as such in the next step.

LC/MS (m/z, MH+): 591

### Step 2: Methyl (R)-6-(2-(ethyl(4-(2-(ethylamino)ethyl)benzyl)amino)-4-methoxyphenyl)-5,6,7,8-tetrahydronaphthalene-2-carboxylate

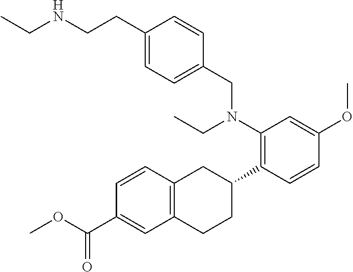 [SMILES: CCNCCc1ccc(CN(CC)c2cc(OC)ccc2[C@@H]2CCc3cc(C(=O)OC)ccc3C2)cc1]

Step 2 of Example 3 was prepared following a similar procedure to that of step 2 of Example 2 from (R)-6-(2-(ethyl(4-(2-(ethylamino)ethyl)benzyl)amino)-4-methoxyphenyl)-5,6,7,8-tetrahydronaphthalen-2-yl trifluoromethanesulfonate to give 27 mg (9%) of methyl (R)-6-(2-(ethyl(4-(2-(ethylamino)ethyl)benzyl)amino)-4-methoxyphenyl)-5,6,7,8-tetrahydronaphthalene-2-carboxylate.

LC/MS (m/z, MH+): 501

### Step 3: (R)-6-(2-(Ethyl(4-(2-(ethylamino)ethyl)benzyl)amino)-4-methoxyphenyl)-5,6,7,8-tetrahydronaphthalene-2-carboxylic acid

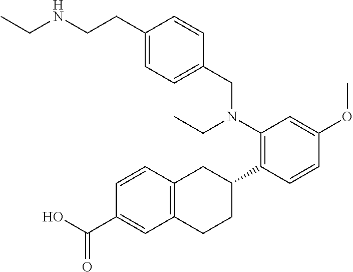 [SMILES: CCNCCc1ccc(CN(CC)c2cc(OC)ccc2[C@@H]2CCc3cc(C(=O)O)ccc3C2)cc1]

Step 3 of Example 3 was prepared following a similar procedure to that of step 4 of Example 1 from methyl (R)-6-(2-(ethyl(4-(2-(ethylamino)ethyl)benzyl)amino)-4-methoxyphenyl)-5,6,7,8-tetrahydronaphthalene-2-carboxylate to give 25 mg (98%) of (R)-6-(2-(ethyl(4-(2-(ethylamino)ethyl)benzyl)amino)-4-methoxyphenyl)-5,6,7,8-tetrahydronaphthalene-2-carboxylic acid.

The compounds according to Table 1 above were subjected to pharmacological tests for determining their degradation effects on estrogen receptors.

### Test: Estrogen Receptor Degradation Activity

Said test involves measuring the in vitro degradation activity of the compounds of the Table 1.

The measurements of the degradation activities were made using a breast cancer cell ERα in cell western assay as described hereunder.

MCF7 cells (ATCC) were seeded in 384 wells microplate (collagen coated) at a concentration of 10000 cells/30 μL per well in red phenol free MEM alpha medium (invitrogen) containing 5% charcoal dextran striped FBS. The following day, 9 points serial 1:5 dilution of each compound was added to the cells in 2.5 μL at final concentrations ranging from 0.3-0.0000018 μM (in Table 2), or 0.1 μM for fulvestrant (using as positive control). At 4 hours post compound addition the cells were fixed by adding 25 μL of formalin (final concentration 5% formalin containing 0.1% triton) for 10 minutes at room temperature and then washed twice with PBS. Then, 50 μL of LI-COR blocking buffer containing 0.1% Triton was added to plate for 30 minutes at room temperature. LI-COR blocking buffer was removed and cells were incubated overnight at cold room with 50 μL anti-ER rabbit monoclonal antibody (Thermo scientific MA1-39540) diluted at 1:1000 in LI-COR blocking buffer containing 0.1% tween-20. Wells which were treated with blocking buffer but no antibody were used as background control. Wells were washed twice with PBS (0.1% tween-20) and incubated at 37° C. for 60 minutes in LI-COR (0.1% tween-20) containing goat anti-rabbit antibody Alexa 488 (1:1000) and Syto-64 a DNA dye (2 μM final concentration). Cells were then washed 3 times in PBS and scanned in ACUMEN explorer (TTP-Labtech). Integrated intensities in the green fluorescence and red fluorescence were measured to determine the levels of ERα and DNA respectively.

The degradation activity with respect to estrogen receptors in this test is given by the concentration which degrades 50% of the estrogen receptor (or IC₅₀) in nM.

The % of ERα levels decrease were determined as follows: % inhibition=100*(1-(sample-fulvestrant: DMSO-fulvestrant)).

The Table 2 below indicates the estrogen receptor degradation activity results for the compounds of Table 1 tested at 0.3 μM, and demonstrates that said compounds have a significant degradation activity on estrogen receptors.

TABLE 2

| | | % |
|---|---|---|
| | Degradation | Degradation |
| Compound No. | IC₅₀ (nM) | At 0.3 μM |
| 1 | 136 | 51 |
| 2 | 196 | 62 |
| 3 | 252 | 49 |

It is therefore apparent that the tested compounds have degradation activities for estrogen receptors, with IC50 less than 1 μM and with degradation levels greater than 50%. The compounds as provided herein can therefore be used for preparing medicaments, especially medicaments which are degraders of estrogen receptors.

Accordingly, also provided herein are medicaments which comprise a compound as defined above, or a pharmaceutically acceptable salt thereof.

Herein are also provided the compounds as defined above, or pharmaceutically acceptable salts thereof, for use as medicines.

Herein are also provided the compounds as defined above, or pharmaceutically acceptable salt thereof, for use in therapy, especially as inhibitors and degraders of estrogen receptors.

Herein are also provided the compounds as defined above, or a pharmaceutically acceptable salts thereof, for use in the treatment of ovulatory dysfunction, cancer, endometriosis, osteoporosis, benign prostatic hypertrophy or inflammation.

A particular aspect is a compound as defined above, or a pharmaceutically acceptable salt thereof, for use in the treatment of cancer.

In an embodiment, the cancer is a hormone dependent cancer.

In another embodiment, the cancer is an estrogen receptor dependent cancer, particularly the cancer is an estrogen receptor a dependent cancer.

In another embodiment, the cancer is selected from breast, ovarian, endometrial, prostate, uterine, cervical and lung cancer, or a metastasis thereof.

In another embodiment, the metastasis is a cerebral metastasis.

In another embodiment, the cancer is breast cancer. Particularly, the breast cancer is an estrogen receptor positive breast cancer (ERα positive breast cancer).

In another embodiment, the cancer is resistant to anti-hormonal treatment.

In a further embodiment, the compound as provided herein is as used as single agent or in combination with other agents such as CDK4/6, mTOR or PI3K inhibitors.

According to another aspect, herein is provided a method of treating the pathological conditions indicated above, comprising administering to a subject in need thereof a therapeutically effective amount of a compound as provided herein, or a pharmaceutically acceptable salt thereof. In an embodiment of this method of treatment, the subject is a human.

Herein is also provided the use of a compound as defined above, or a pharmaceutically acceptable salt thereof, for the manufacture of a medicament useful in treating any of the pathological conditions indicated above, more particularly useful in treating cancer.

Herein are also provided the pharmaceutical compositions comprising as active principle a compound as defined above. These pharmaceutical compositions comprise an effective dose of at least one compound as defined above, or a pharmaceutically acceptable salt thereof, and also at least one pharmaceutically acceptable excipient.

The said excipients are selected, in accordance with the pharmaceutical form and method of administration desired, from the customary excipients, which are known to a person skilled in the art.

In the pharmaceutical compositions for oral, sublingual, subcutaneous, intramuscular, intravenous, topical, local, intra-tracheal, intranasal, transdermal or rectal administration, the active principle as defined above, or its base, acid, zwitterion or salt thereof, may be administered in a unit administration form, in a mixture with conventional pharmaceutical excipients, to animals and to human beings for the treatment of the above disorders or diseases.

The unit administration forms appropriate include oral forms such as tablets, soft or hard gel capsules, powders, granules and oral solutions or suspensions, sublingual, buccal, intra-tracheal, intra-ocular and intra-nasal administration forms, forms for inhalative, topical, transdermal, subcutaneous, intra-muscular or intravenous administration, rectal administration forms and implants. For topical application it is possible to use the compounds as provided herein in creams, gels, ointments or lotions.

As an example, a unit administration form of a compound as provided herein in tablet form may comprise the following components:

| | | |
|---|---|---|
| Compound as provided herein | 50.0 | mg |
| Mannitol | 223.75 | mg |
| Sodium croscarmellose | 6.0 | mg |
| Corn starch | 15.0 | mg |
| Hydroxypropylmethylcellulose | 2.25 | mg |
| Magnesium stearate | 3.0 | mg |

There may be particular cases in which higher or lower dosages are appropriate. According to usual practice, the dosage that is appropriate for each patient is determined by the doctor according to the mode of administration and the weight and response of the said patient.

1. A compound, or a pharmaceutically acceptable salt thereof, in particular hydrochloride salt thereof, characterized in that said compound is selected from the following compounds:
4-(4-((1-(3-fluoropropyl)azetidin-3-yl)oxy)benzoyl)-3-(4-(trifluoromethyl)phenyl)quinoline-7-carboxylic acid (1),
3-(2-fluoro-4-(trifluoromethyl)phenyl)-4-(4-(2-(3-(fluoromethyl)azetidin-1-yl)ethoxy)benzoyl)quinoline-7-carboxylic acid (2), and
(R)-6-(2-(ethyl(4-(2-(ethylamino)ethyl)benzyl)amino)-4-methoxyphenyl)-5,6,7,8-tetrahydronaphthalene-2-carboxylic acid (3).

2. The compound according to claim 1, or a pharmaceutically acceptable salt thereof, in particular hydrochloride salt thereof, selected from the following compounds:
4-(4-((1-(3-fluoropropyl)azetidin-3-yl)oxy)benzoyl)-3-(4-(trifluoromethyl)phenyl)quinoline-7-carboxylic acid (1), and
(R)-6-(2-(ethyl(4-(2-(ethylamino)ethyl)benzyl)amino)-4-methoxyphenyl)-5,6,7,8-tetrahydronaphthalene-2-carboxylic acid (3).

3. Compound, or any of its pharmaceutically acceptable salt, selected from

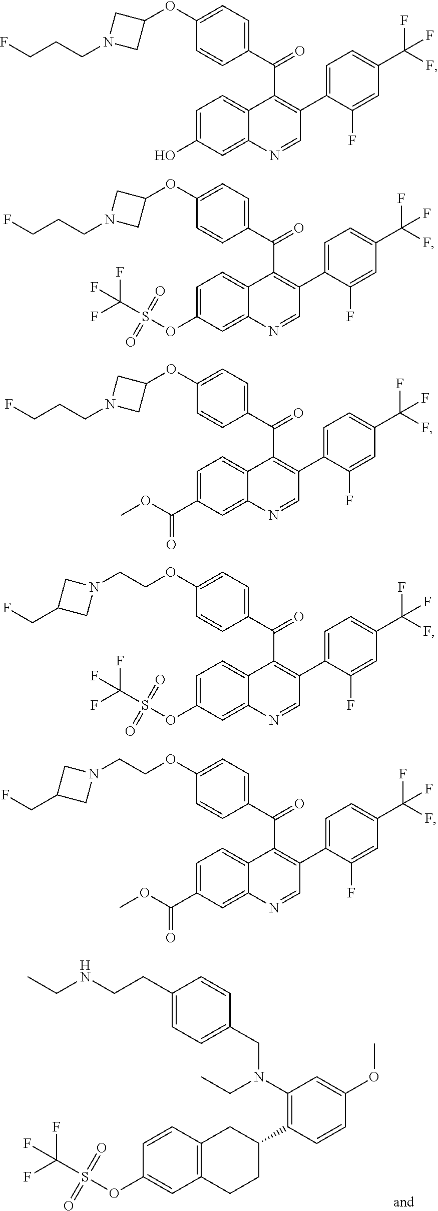

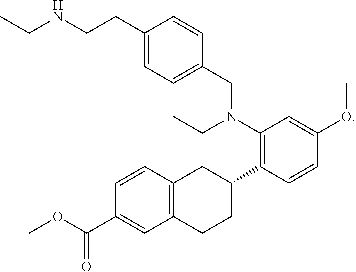 [SMILES: CCNCCc1ccc(CN(CC)c2cc(OC)ccc2[C@@H]2CCc3cc(C(=O)OC)ccc3C2)cc1]

4. A medicament, characterized in that it comprises a compound according to claim 1 or 2, or a pharmaceutically acceptable salt thereof.

5. A pharmaceutical composition, characterized in that it comprises a compound according to claim 1 or 2, or a pharmaceutically acceptable salt thereof, and at least one pharmaceutically acceptable excipient.

6. A compound according to claim 1 or 2, or a pharmaceutically acceptable salt thereof, for use as an inhibitor and degrader of estrogen receptors.

7. A compound according to claim 1 or 2, or a pharmaceutically acceptable salt thereof, for use in the treatment of ovulatory dysfunction, cancer, endometriosis, osteoporosis, benign prostatic hypertrophy or inflammation.

8. A compound for use according to claim 6, or a pharmaceutically acceptable salt thereof, for use in the treatment of cancer.
